(19) United States
(12) Patent Application Publication
(10) Pub. No.: US 2014/0257148 A1
(43) Pub. Date: Sep. 11, 2014

(54) ULTRASOUND LITHOTRITE

(71) Applicant: FUZHOU SECURE OPTIC & ELECTRONIC CO., LTD., Fuzhou (CN)

(72) Inventor: Yebing Jie, Fuzhou (CN)

(21) Appl. No.: 14/350,486

(22) PCT Filed: Nov. 1, 2012
(86) PCT No.: PCT/CN2012/083924

§ 371 Date: Apr. 8, 2014

(30) Foreign Application Priority Data
Nov. 1, 2011 (CN) 201110339547.0

(51) Int. Cl. A61B 17/225

(52) U.S. Cl. CPC A61B 17/225
USPC 601/4

(57) ABSTRACT

An ultrasound lithotrite comprises a handle main body (1) and a hand-held portion (2) connected to the handle main body (1), and an ultrasound generator (15) is disposed in the handle main body (1). A first channel (111) is disposed on an internal central axis of the handle main body (1), a second channel (211) is disposed on the internal central axis of the hand-held portion (2), and the first channel (111) and the second channel (211) are on the same straight line. The ultrasound lithotrite further comprises an ultrasound forceps (3) connected to the ultrasound generator (15) and passed through the first channel (111) and the second channel (211) for gripping a stone. The back end of the ultrasound forceps (3) is provided with a horizontal limiting slot (32), and the ultrasound forceps (3) are fixed to the hand-held portion (2) through the horizontal limiting slot (32) and are moved horizontally along the first channel (111) and the second channel (211) with the hand-held portion (2).

### TECHNICAL FIELD

The present invention relates to medical device field, in particular to an ultrasound lithotrite.

### BACKGROUND

In the days with rapid economic development, people's life rhythm is accelerated, dietary configuration is changed, a lot of people sit and work at the office for a long time and is lack of exercise, and some people often can't drink water for several tunes a day or even don't drink water because of work stress. Therefore the incidence of lithiasis shows the increasing trend more and more. Thus the requirement for medical device applied to lithotripsy increases day by day. Especially the in vivo ultrasonic lithotripsy hurts the human body less and become the first choice for lithotripsy. However, upon the lithotripsy procedure it is often difficult to locate the stone due to the slide of the stone. Chinese patent applied with publication No. CN101461732 discloses an ultrasound lithotripter using a stone-breaking amplitude changing rod for breaking up the stone through ultrasonic lithotripsy. However, the ultrasound lithotripter with the above structure can't directly locate the stone, thus the stone still slides in the body, and it is easily leads to injury to the other healthy organ caused by the ultrasound lithotripter, which finally leads to a medical malpractice and greatly extends the time for breaking up the stone.

### SUMMARY

The present invention aims at solving the defect that the ultrasound lithotripter in the prior art can't completely locate the stone in the body, thus the stone slides in the body and leads to injury to the other healthy organ caused by the ultrasound lithotripter, which finally leads to a medical malpractice and greatly extends the time for breaking up the stone, and the present invention provides an ultrasound lithotrite for gripping the stone in the body.

The technical solution of the present invention for solving the technical problem is: an ultrasound lithotrite comprising a handle main body and a hand-held portion connected to the handle main body, and an ultrasound generator being disposed in the handle main body, wherein a first channel is disposed on an internal central axis of, the handle main body, a second channel is disposed on the internal central axis of the hand-held portion, and the first channel and the second channel are on the same straight line, the ultrasound lithotrite further includes an ultrasound forceps connected to the ultrasound generator and passed through the first channel and the second channel for gripping a stone in a body, a front end of the ultrasound forceps is provided with a pair of naturally opened gripping sheets, a back end of the ultrasound forceps is provided with a horizontal limiting slot, and the ultrasound forceps are fixed to the hand-held portion through the horizontal limiting slot and are moved horizontally along the first channel and the second channel with the hand-held portion.

Specifically, the handle main body includes a handle front cover, a handle housing and a handle connecting head for connecting the handle housing and the hand-held portion, the handle front cover, the handle housing and the handle connecting head are movably connected in sequence, and the ultrasound generator is disposed in the handle housing.

Furthermore, the handle front cover is threadedly connected with the handle housing, the handle housing is threadedly connected with the handle connecting head.

Furthermore, the handle main body further includes an ultrasound probe rod movably connected with the ultrasound generator, the ultrasound probe rod is a slender rod, the slender rod is provided with a third channel for the ultrasound forceps to be passed through, the third channel, the first channel and the second channel are on the same straight line, and the gripping sheets of the ultrasound forceps are folded in the third channel.

Furthermore, the handle connecting head is provided with a side adsorption connecting seat connected with the first channel and communicated with an adsorber.

Specifically, the hand-held portion includes a hand-held main body connected with the handle main body and a elastic U-shaped hand-held sheet connected with the hand-held main body and movable in the hand-held main body along the second channel, and the hand-held main body is provided with a hollow groove.

Furthermore, the hollow groove of the hand-held main body is provided with a limiting component, the limiting component includes a sliding block slid horizontally along the central axis of the hand-held main body, the sliding block is provided with a fourth channel for the ultrasound forceps to be passed through and a slot vertical to the fourth channel, the slot is provided with a button for being inserted to the horizontal limiting slot, a reset spring is disposed between the button and the bottom of the slot, one end of the elastic U-shaped hand-held sheet is fixedly connected with the hand-held main body, and the other end of the elastic U-shaped hand-held sheet is fixedly connected with the sliding block.

Furthermore, the button is a cylinder, the cylinder is provided with a U-shaped through hole matched with the horizontal limiting slot of the ultrasound forceps, the ultrasound forceps is disposed in the U-shaped through hole, the bottom of the cylinder is provided with a through hole for accommodating the reset spring therein, and the through hole is communicated with the U-shaped through hole.

Furthermore, a buffering component is disposed between the sliding block and the hand-held main body, the buffering component is disposed on a side of the sliding block, the ultrasound forceps is passed through the center of the buffering component.

The beneficial effect of the present invention is that: the ultrasound lithotrite of the present invention is provided with a ultrasound forceps for gripping the stone in the body, the ultrasound forceps is used to stably grip the stone sliding in the body and after grip process the gripped stone is located and broken up; and an ultrasound generator is disposed in the ultrasound forceps, the ultrasound generated by the ultrasound generator transmits to the stone gripped by the front end of the ultrasound forceps through the ultrasound forceps, then the stone is shattered through the vibration frequency of the ultrasound, which, prevents the ultrasound from damaging the other healthy organs in the body, increases the efficiency of breaking up the stone and saves time, and is capable of finishing a targeted break-up for stone in the body one by one, to minimize the pain of patient; meanwhile, the ultrasound forceps may be taken out completely, and the ultrasound forceps is connected with an absorber through a side adsorption connecting seat, then the ultrasound forceps is extended to the patient body and the broken small stone in the patient body may be sucked out, therefore the stone is drained out of the patient body, and using the same mechanism to break and attract the stone may be achieved, thereby reducing treatment time for the patient.

### BRIEF DESCRIPTION OF THE DRAWINGS

FIG. 1 is a schematic sectional view of an ultrasound lithotrite according to an embodiment of the present invention (the gripping sheets are folded);

FIG. 2 is a schematic sectional view of the ultrasound lithotrite according to the embodiment of the present invention (the gripping sheets are in natural state);

FIG. 3 is an explosive view of the ultrasound lithotrite according to the embodiment of the present invention;

FIG. 4 is a schematic perspective view of a hand-held main body of the ultrasound lithotrite according to the embodiment of the present invention;

FIG. 5 is a schematic perspective view of a sliding block of the ultrasound lithotrite according to the embodiment of the present invention;

FIG. 6 is a schematic perspective view of a sliding block of the ultrasound lithotrite according to the embodiment of the present invention.

In the figures: 1—handle main body, 11—handle front cover, 111—first channel, 12—handle housing, 121—handle wire out seat, 13—handle connecting head, 131—side adsorption connecting seat, 14—ultrasound probe rod, 15—ultrasound generator, 2—hand-held portion, 21—hand-held main body, 211—second channel, 212—hollow groove, 22—elastic U-shaped hand-held sheet, 23—limiting component, 231—sliding block , 2311—fourth channel, 2312—slot, 232—button, 2321—U-shaped through hole, 2322—through hole on the bottom of the button, 233—reset spring, 234—buffering component, 3—ultrasound forceps, 31—gripping sheet, 32—horizontal limiting slot of ultrasound forceps

### DETAILED DESCRIPTION OF THE PREFERRED EMBODIMENTS

In order to make purpose, technical solution and advantages of the present invention more clearly be understood, the present invention will be described in further detail in conjunction with embodiments and with reference to the accompanying drawings. It should be understood that the embodiments described herein are only used for explaining the present invention, and are not intended to limit the present invention.

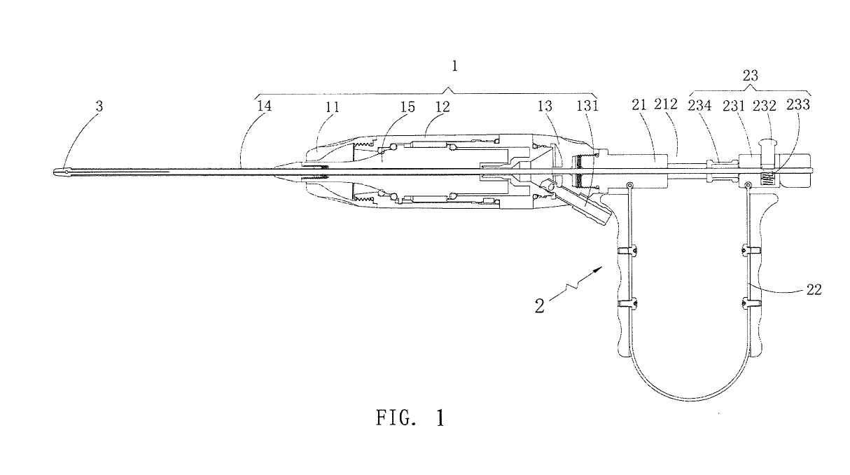

FIG. 1 to FIG. 3 show an ultrasound lithotrite of the present invention. The ultrasound lithotrite includes a handle main body 1 and a hand-held portion 2 connected to the handle main body 1, and an ultrasound generator 15 is disposed in the handle main body 1. The ultrasound lithotrite of the present invention may break up the stone in the patient body through the ultrasound with certain frequency generated by the ultrasound generator 15. Specifically, a first channel 111 is defined in the internal central axis of the handle main body 1 of the present invention, a second channel 211 is defined in the internal central axis of the hand-held portion 2, and the first channel 111 and the second channel 211 are on the same straight line. That is, the handle main body 1 and the hand-held portion are coaxial. The most important thing of the present invention is that the ultrasound lithotrite further includes an ultrasound forceps 3 connected to the ultrasound generator 15 and passed through the first channel 111 and the second channel 211 for gripping a stone in the body. Therefore the ultrasound forceps 3 is passed through the handle main body 1 and the hand-held portion 2, the handle main body 1 and the hand-held portion 2 support the ultrasound forceps 3 and limit the radial motion of the ultrasound forceps 3. Also, the front end of the ultrasound forceps 3 is provided with a pair of naturally opened gripping sheets 31, the back end of the ultrasound forceps 3 is provided with a horizontal limiting slot 32, and the ultrasound forceps 3 are fixed to the hand-held portion 2 through the horizontal limiting slot 32 and are moved horizontally along the first channel and the second channel with the hand-held portion 2.

Specifically, referring to FIG. 1 to FIG. 3 again, the handle main body 1 includes a handle front cover 11, a handle housing 12 and a handle connecting head 13 for connecting the handle housing 12 and the hand-held portion 2, the handle front cover 11, the handle housing 12 and the handle connecting head 13 are movably connected in sequence, and the ultrasound generator 15 is disposed in the handle housing 12. The handle front cover 11 is threadedly connected with the handle housing 12, the handle housing 12 is threadedly connected with the handle connecting head 13. The handle main body of the ultrasound lithotrite of the present invention includes a front section, a middle section and a back section, the ultrasound generator 15 is fixed in the handle housing of the middle section and three ends of the ultrasound generator 15 are connected by using the screw thread. Therefore the structure of handle main body is simple, easy to be disassembled and convenient to be washed or replaced a component.

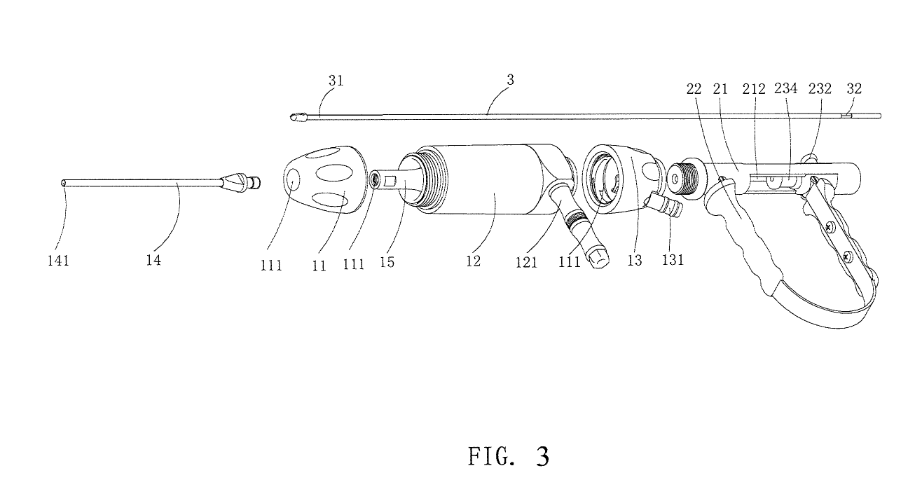

Furthermore, referring to FIG. 3, the handle main body 1 further includes an ultrasound probe rod 14 movably connected with the ultrasound generator 15, the ultrasound probe rod 14 is a slender rod, the slender rod is provided with a third channel 141 for the ultrasound forceps 3 to be passed through, the third channel 141, the first channel 111 and the second channel 211 are on the same straight line, a pair of gripping sheets 31 of the ultrasound forceps are folded in the third channel 141. And the handle connecting head 13 is provided with a side adsorption connecting seat 131 connected with the first channel and communicated with an adsorber (not shown in the figure). For the ultrasound lithotrite of the present invention, the ultrasound forceps 3 may be taken out completely, then the ultrasound forceps may be extended to the patient body and the broken small stone in the patient body may be sucked out one by one through the adsorber. The adsorber is connected with the side adsorption connecting seat 131 on the handle main body 13 and configured to provide an attraction force. The small stone in the patient body passes through the third channel 141 of the ultrasound probe rod 14 and enters the first channel 111 of the handle main body 1, and finally is drained out from the side adsorption connecting seat 131. The ultrasound probe rod 14 of the present invention is also configured to fold a pair of gripping sheets 31 on the front end of the ultrasound forceps 3. Because the elastic sheet 13 is open in the natural state, the elastic sheet 13 is disposed in the ultrasound probe rod 14 to be folded. When the elastic sheet 13 is used to grip, it is just needed to horizontally push the ultrasound forceps 131 forward, and the elastic sheet 13 disposed in the ultrasound probe rod 14 is horizontally pushed out, and the open elastic sheet 31 is configured to grip the stone in the patient body.

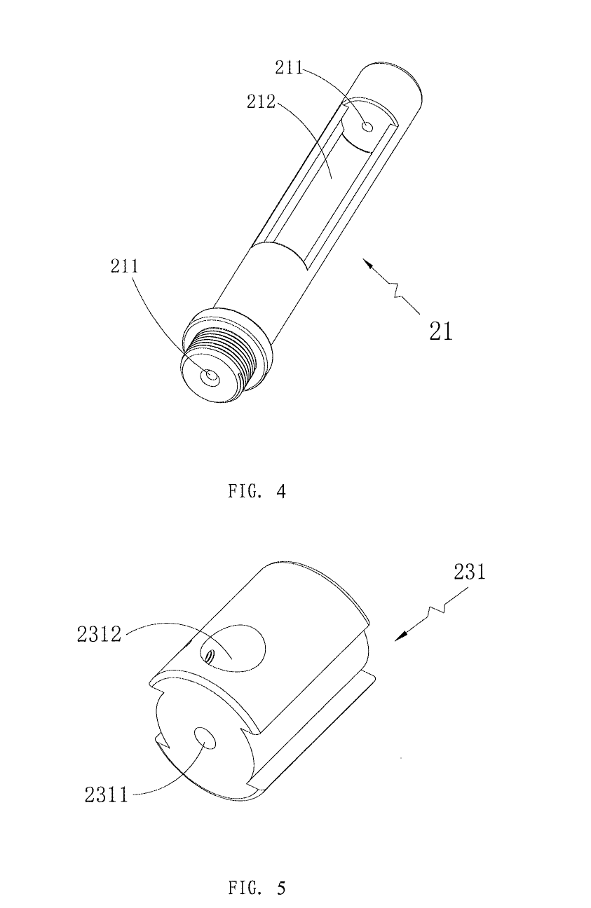

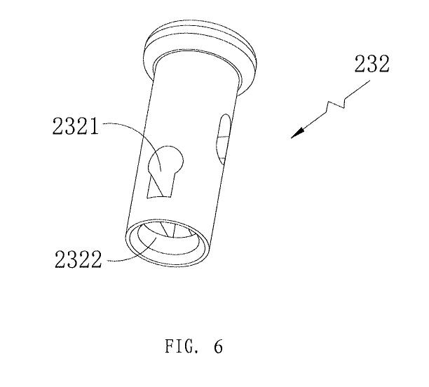

Specifically, referring to FIG. 1 to FIG. 3 again, the hand-held portion 2 includes a hand-held main body 21 connected with the handle main body 2 and an elastic U-shaped hand-held sheet 22 connected with the hand-held main body 21 and movable in the hand-held main body 21 along the second channel 211, the hand-held main body 21 is provided with a hollow groove 212. The elastic U-shaped hand-held sheet 22 is a leaf spring, when the elastic U-shaped hand-held sheet 22 is clenched by the hand, a U-shaped opening of the elastic U-shaped hand-held sheet 22 is folded, when the elastic U-shaped hand-held sheet 22 is not clenched by the hand, the elastic U-shaped hand-held sheet 22 recovers. Referring to FIG. 4, the hollow groove 212 of the hand-held main body 21 is provided with a limiting component 23, the limiting component 23 includes a sliding block 231 slid horizontally along the central axis of the hand-held main body 21, one end of the elastic U-shaped hand-held sheet 22 is fixedly connected with the hand-held main body 2, and the other end of the elastic U-shaped hand-held sheet 22 is fixedly connected with the sliding block 231. Referring to FIG. 5, the sliding block 231 is provided with a fourth channel 2311 for the ultrasound forceps 3 to be passed through and a slot 2312 vertical to the fourth channel 2311, the slot 2312 is provided with a button 232 for being inserted to the horizontal limiting slot 32, and a reset spring 233 is disposed between the button 232 and the bottom of the slot 2312. Referring to FIG. 6, the button 232 is a cylinder, the cylinder is provided with a U-shaped through hole 2312 matched with the horizontal limiting slot 32 of the ultrasound forceps 3, the ultrasound forceps 3 is disposed in the U-shaped through hole 2321, the bottom of the cylinder is provided with a through hole for accommodating the reset spring, the through hole is communicated with the U-shaped through hole. A buffering component 234 is disposed between the sliding block 231 and the hand-held main body 21, the buffering component is disposed on a side of the sliding block, and the ultrasound forceps is passed through the center of the buffering component.

The ultrasound forceps 3 of the ultrasound lithotrite of the present invention is passed through the handle main body and the hand-held portion, and is fixed in the U-shaped through hole 2321 of the button 232 on the sliding block 231 of the hand-held portion 2 through the horizontal limiting slot 32 in the back end of the ultrasound forceps 3. When the horizontal limiting slot 32 of the ultrasound forceps 3 is passed through the U-shaped through hole 2321 of the button 232, the reset spring 233 jacks up the button 232, the horizontal limiting slot 32 is clamped to the bottom of the U-shaped through hole, then the ultrasound forceps 3 is fixed to the button 232 in the horizontal direction, and the button 232 is fixed to the sliding block 231. Therefore the ultrasound forceps follows the sliding block 231 and is horizontally moved in the hollow groove 212 of the hand-held main body. And a hand-held part of the ultrasound forceps 3 is formed by fixedly connecting one end of the elastic U-shaped hand-held sheet 22 to a fixing end of the hand-held portion 2 and fixing the other end of the elastic U-shaped hand-held sheet 22 to the sliding block 231 in the hollow groove 212 of the hand-held portion 2, and the hollow groove 212 functions as limitation. Therefore the sliding block 231 follows the elastic U-shaped hand-held sheet 22 and is moved in the hollow groove 212, that is, the ultrasound forceps 3 fixed to the sliding block 231 follows a U-shaped opening of the elastic U-shaped hand-held sheet 22 and is horizontally moved along the central axis of the handle main body. One end of the sliding block 231 is provided with a buffering component 234, to avoid the elastic U-shaped hand-held sheet 22 being held over force, the sliding block 234 colliding the other end of hollow groove 21 and the ultrasound forceps 3 extending forward too much in the horizontal direction.

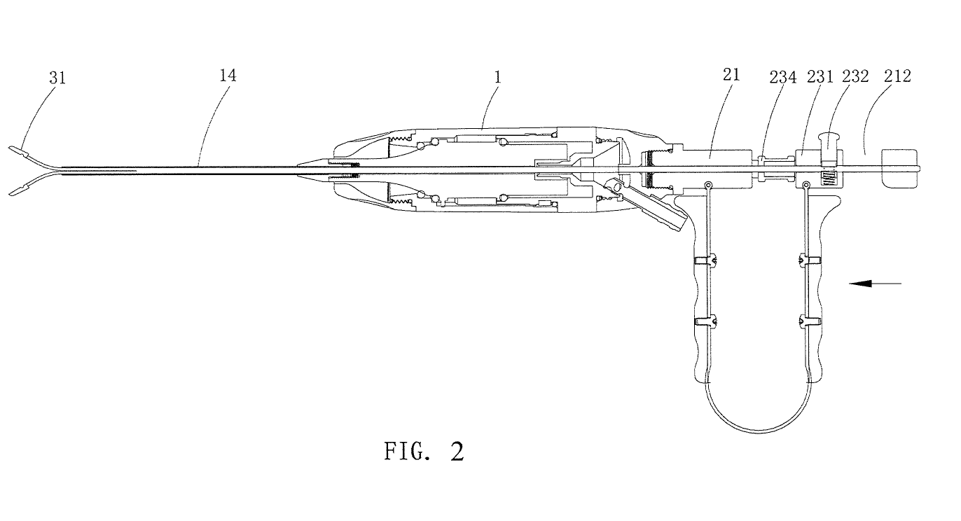

When using the ultrasound lithotrite of the present invention to break up the stone, firstly, as shown in FIG. 1, it is confirmed that the ultrasound forceps 3 is fixed to the ultrasound lithotrite through the first channel 111 and the second channel 211 and may follow the sliding block 234 and horizontally slide along a central axis. When the elastic U-shaped hand-held sheet 22 is in a release state, the sliding block 231 presses against the black end of the hollow groove 212 of the hand-held portion 2, and a pair of gripping sheet 31 of the forceps 3 is folded in the ultrasound probe rod 14. Then, the elastic U-shaped hand-held sheet 22 is held, the ultrasound lithotrite of the present invention is extended to the body the patient and gets close to the stone of the patient as far as possible. Then the elastic U-shaped hand-held sheet 22 is applied a force, as shown in FIG. 2, the sliding block 232 is moved forward and brings the ultrasound forceps 3 to move forward, the gripping sheet 31 extends out from the fourth channel 141 of the ultrasound probe rod 14 and is in an unfolding state. Then the gripping sheet 31 is used to contact the stone, when the gripping sheet 31 contacts the stone, stop applying the force, and the elastic U-shaped hand-held sheet 22 is automatically reset and brings the sliding block to move backward, thereby bringing the ultrasound forceps to move backward. Then the gripping sheet is folded gradually, thus to grip the stone of the patient, therefore, the stone is fixed to the ultrasound lithotrite of the present invention and doesn't slide. Finally, a handle wire out seat of the handle housing 12 controls the ultrasound generator 15 generate the ultrasound, the ultrasound transmits to the gripping sheet 31 on the front end through the ultrasound forceps 3, therefore, the stone is broken through ultrasound with certain frequency. The special ultrasound forceps of the present invention stably grips the stone sliding in the body, then the vibration frequency of the ultrasound breaks up the stone in the body, thus it avoids the ultrasound damaging the other healthy organ in the body and meanwhile increases the efficiency of breaking up the stone and save time. It is capable of finishing a targeted break-up for stone in the body one by one, thereby minimize the pain of patient.

Meanwhile, the present invention may also take the stone out. Firstly the button 232 of the hand-held portion 2 is pressed, the horizontal limiting slot of the ultrasound forceps slides to the top of the U-shaped through hole in the button, then the ultrasound forceps may be completely taken out of the ultrasound lithotrite. Then the ultrasound probe rod 14 of the ultrasound lithotrite of the present invention is extended to the patient body. The side adsorption connecting seat 131 of the handle connecting head 13 is connected to the adsorber which provides a negative pressure, and the third channel of the ultrasound probe rod 14 is communicated with the first channel 111 of the handle main body, and the negative pressure provided by the adsorber functions as adsorption. The ultrasound probe rod 14 sucks the small stone out of the body, therefore, the stone is drained out of the patient body. Using the same mechanism to break and attract the stone may be achieved through the ultrasound lithotrite of the present invention, therefore, treatment time for the patient is reduced.

The foregoing are only preferred embodiments of the present invention, and are not intended to limit the present invention. Within the spirit and principles of the present invention, any modifications, equivalent replacement, improvement, etc., should be included within the scope of protection of the present invention.

What is claimed is:

1. An ultrasound lithotrite comprising a handle main body and a hand-held portion connected to the handle main body, and an ultrasound generator being disposed in the handle main body, wherein a first channel is disposed on an internal central axis of the handle main body, a second channel is disposed on the internal central axis of the hand-held portion, and the first channel and the second channel are on the same straight line, the ultrasound lithotrite further comprises an ultrasound forceps connected to the ultrasound generator and passed through the first channel and the second channel for gripping a stone in a body, a front end of the ultrasound forceps is provided with a pair of naturally opened gripping sheets, a back end of the ultrasound forceps is provided with a horizontal limiting slot, and the ultrasound forceps are fixed to the hand-held portion through the horizontal limiting slot and are moved horizontally along the first channel and the second channel with the hand-held portion.

2. The ultrasound lithotrite of claim 1, wherein the handle main body comprises a handle front cover, a handle housing and a handle connecting head for connecting the handle housing and the hand-held portion, the handle front cover, the handle housing and the handle connecting head are movably connected in sequence, and the ultrasound generator is disposed in the handle housing.

3. The ultrasound lithotrite of claim 2, wherein, the handle front cover is threadedly connected with the handle housing, the handle housing is threadedly connected with the handle connecting head.

4. The ultrasound lithotrite of claim 2, wherein, the handle main body further comprises an ultrasound probe rod movably connected with the ultrasound generator, the ultrasound probe rod is a slender rod, the slender rod is provided with a third channel for the ultrasound forceps to be passed through, the third channel, the first channel and the second channel are on the same straight line, and the gripping sheets of the ultrasound forceps are folded in the third channel.

5. The ultrasound lithotrite of claim 2, wherein, the handle connecting head is provided with a side adsorption connecting seat connected with the first channel and communicated with an adsorber.

6. The ultrasound lithotrite of claim 1, wherein, the hand-held portion comprises a hand-held main body connected with the handle main body and an elastic U-shaped hand-held sheet connected with the hand-held main body and movable in the hand-held main body along the second channel, and the hand-held main body is provided with a hollow groove.

7. The ultrasound lithotrite of claim 6, wherein, the hollow groove of the hand-held main body is provided with a limiting component, the limiting component comprises a sliding block slid horizontally along a central axis of the hand-held main body, the sliding block is provided with a fourth channel for the ultrasound forceps to be passed through and a slot vertical to the fourth channel, the slot is provided with a button for being inserted to the horizontal limiting slot, a reset spring is disposed between the button and the bottom of the slot, one end of the elastic U-shaped hand-held sheet is fixedly connected with the hand-held main body, and the other end of the elastic U-shaped hand-held sheet is fixedly connected with the sliding block.

8. The ultrasound lithotrite of claim 7, wherein, the button is a cylinder, the cylinder is provided with a U-shaped through hole matched with the horizontal limiting slot of the ultrasound forceps, the ultrasound forceps is disposed in the U-shaped through hole, the bottom of the cylinder is provided with a through hole for accommodating the reset spring therein, and the through hole is communicated with the U-shaped through hole.

9. The ultrasound lithotrite of claim 7, wherein, a buffering component is disposed between the sliding block and the hand-held main body, the buffering component is disposed on a side of the sliding block, and the ultrasound forceps is passed through the center of the buffering component.
